(19) United States
(12) Plant Patent Application Publication
(10) Pub. No.: US 2019/0307041 P1
(43) Pub. Date: Oct. 3, 2019

(54) Artichoke variety named 'PAGA G-1'

(50) ;

(71) Applicant: Michael R. SCATTINI, Salinas, CA (US)

(72) Inventor: Michael R. SCATTINI, Salinas, CA (US)

(21) Appl. No.: 16/501,358

(22) Filed: Mar. 29, 2019

Related U.S. Application Data
(60) Provisional application No. 62/650,988, filed on Mar. 30, 2018.

(51) Int. Cl. A01H 6/14

(52) U.S. Cl. PLT/258

(57) ABSTRACT

A new and distinct variety of artichoke (Cynara cardunculus var. scolymus L.) plant named ‘PAGA G-1’, particularly characterized by its high yield, large primary bud size, large number of secondary and tertiary buds, wide bracts with fleshy base, thick stems, and early maturity, is disclosed.

### CROSS REFERENCE TO RELATED APPLICATION

This application claims the benefit of U.S. Prov. App. No. 62/650,988, filed Mar. 30, 2018, which is hereby incorporated by reference in its entirety.

Latin name: Botanical classification: Cynara cardunculus L. var. scolymus.

Varietal denomination: Varietal denomination of the claimed artichoke variety: ‘PAGA G-1’.

### BACKGROUND OF THE INVENTION

Artichoke (Cynara cardunculus L. var. scolymus), or globe artichoke, is a thistle-like perennial plant native to the Mediterranean region and is a member of the family Asteraceae. Artichoke belongs to the species Cynara cardunculus L. that includes two subspecies/cultivation groups: the cardoon (Cynara cardunculus L. var. altilis) that is cultivated for its edible leaf stems, and the artichoke that is cultivated for its edible immature inflorescence hereinafter referred to as buds, heads, or capitula. In some instances, artichoke is distinguished as Cynara scolymus.

Artichoke is a popular vegetable food source in many regions of the world, with nearly two million tons of artichokes produced annually. The United States produces approximately 40,000 tons of artichokes annually, with California being responsible for nearly 100% of its production, of which about 80% is grown in Monterey County.

Historically, a common perennial artichoke variety that is grown in the United States is ‘Green Globe’, also referred to as an “Heirloom” artichoke. Despite the use of it as a traditional cultivar, ‘Green Globe’ does not produce large numbers of large buds which are most desired by consumers. Further, ‘Green Globe’ exhibits a substantial amount of phenotypic variation as well as protracted and unpredictable harvest intervals.

Thus, there exists a need for improved artichoke varieties having large sized post-primary bud production, programmable and predictable harvest timing, and better uniformity. The present artichoke variety described herein is a product of the efforts to develop such artichoke varieties.

### BRIEF SUMMARY

The present disclosure relates to a new and distinct artichoke variety (Cynara cardunculus L. var. scolymus) which has been denominated as ‘PAGA G-1’. This new artichoke variety exhibits increased bud yield, size and plant vigor compared to ‘Green Globe’, and produces a large primary bud, about 7-10 secondary buds, and about 6-14 tertiary buds per primary reproductive shoot: Up to 8 of the primary and secondary buds are 12-15 inches in circumference. Between 4-5 of the secondary and tertiary buds are 10¾-12½ inches in circumference or larger. The remaining buds are generally between 3-10½ inches in circumference. The plants produce between 1 and 5 reproductive shoots, in addition to the primary reproductive shoot, in the first production cycle. These secondary reproductive shoots produce less marketable sized buds than the primary shoot, although cultural practices such as cutting the primary reproductive shoot from the plant after harvest can materially increase the size and number of buds produced by these secondary shoots.

The new artichoke variety described herein is the product of an artichoke plant improvement program conducted over a number of years. The original parent is the unpatented Cynara cardunculus L. var. scolymus variety ‘Green Globe’, commonly known and cultivated in Castroville, Calif. for more than 90 years. This improvement project began with the objective to develop an artichoke variety with superior bud production. This included selecting for improvements in both commercial quality of buds, as well as overall bud consistency and large size bud production per plant. Additionally, plants were selected based on their predictable and condensed harvest phase.

The new artichoke variety described herein was discovered after several years of isolating and propagating artichoke variety ‘Green Globe’ in Castroville, Calif. From this continual propagation, several selections were identified which exhibited superior yield characteristics as compared to ‘Green Globe’. After identifying the superior quality plants from field conditions, the highest quality plants were selected for induction into laboratory tissue culture reproduction. These tissue-cultured plants were then planted in a test field in Castroville, Calif., where further selections were made among the population. Selections from this field were propagated by tissue culture, with subsequent field selections and tissue culture propagation conducted. The variety described herein as ‘PAGA G-1’ is a final product obtained from these selections and tissue culture propagation cycles.

Artichoke variety ‘PAGA G-1’ was subsequently asexually reproduced via tissue culture propagation and regeneration of artichoke plants from plantlets. The present variety has been found to be stable and reproduce true to type through successive asexual propagations.

‘PAGA G-1’ exhibits the following distinguishing characteristics when grown under normal horticultural practices in Castroville, Calif.:
1. Large bud size;
2. Primary reproductive shoots produce at least 6 secondary buds, and at least 3 tertiary buds;
3. Plants produce an average of 3 reproductive shoots, in addition to the primary reproductive shoot, during the first production cycle;
4. Bracts and receptacles are wide, and thick;
5. Large diameter peduncle; and
6. Early maturity.

### DESCRIPTION OF THE DRAWINGS

The accompanying photographs illustrate the overall appearance of the new artichoke variety ‘PAGA G-1’ showing the colors as true as is reasonably possible with colored reproductions of this type. Colors in the photographs may differ slightly from the color values cited in the detailed botanical description, which accurately describes the color of this variety. The depicted plant and plant parts of the new artichoke variety ‘PAGA G-1’ are from plants that are 5-6 months old, grown in Castroville, Calif.

FIG. 1 shows a large sized capitulum (bud) of ‘PAGA G-1’.

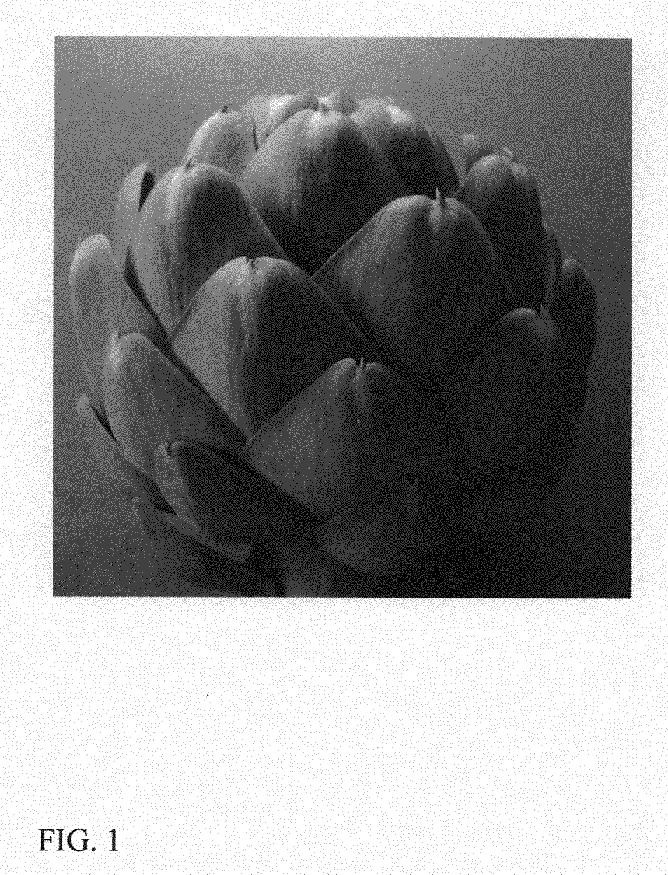

FIG. 2 shows an interior view of a bract of ‘PAGA G-1’.

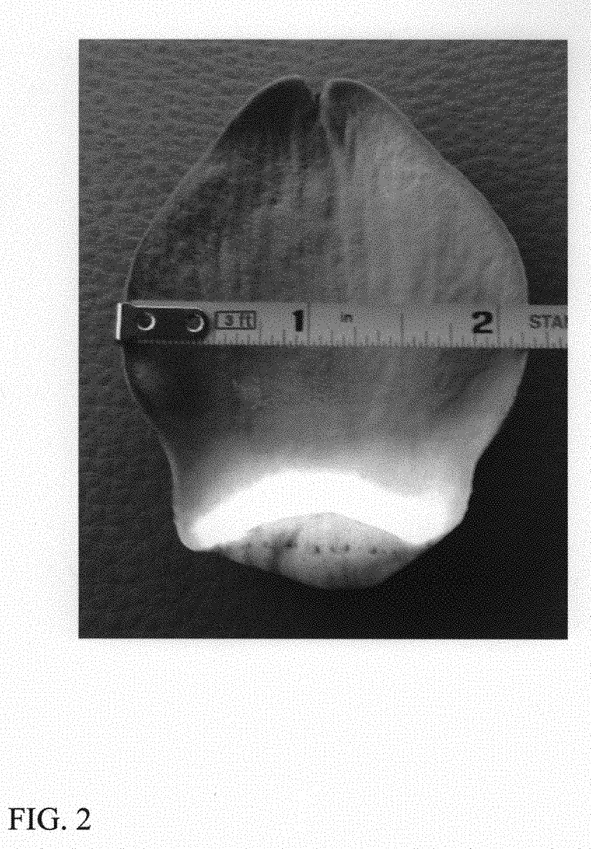

FIG. 3 shows an exterior view of a bract of ‘PAGA G-1’.

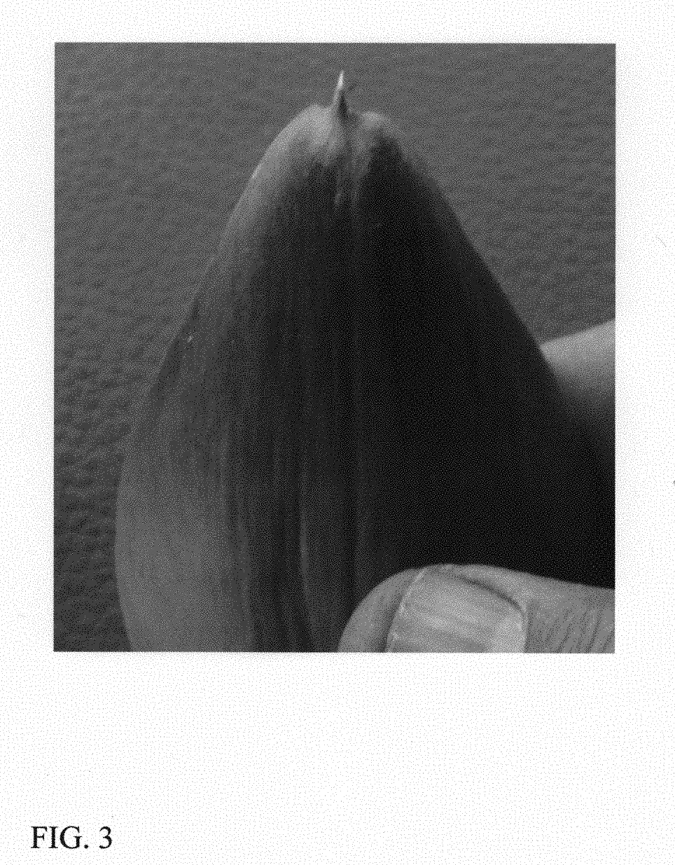

FIG. 4 shows a cross section of a capitulum (bud) of ‘PAGA G-1’.

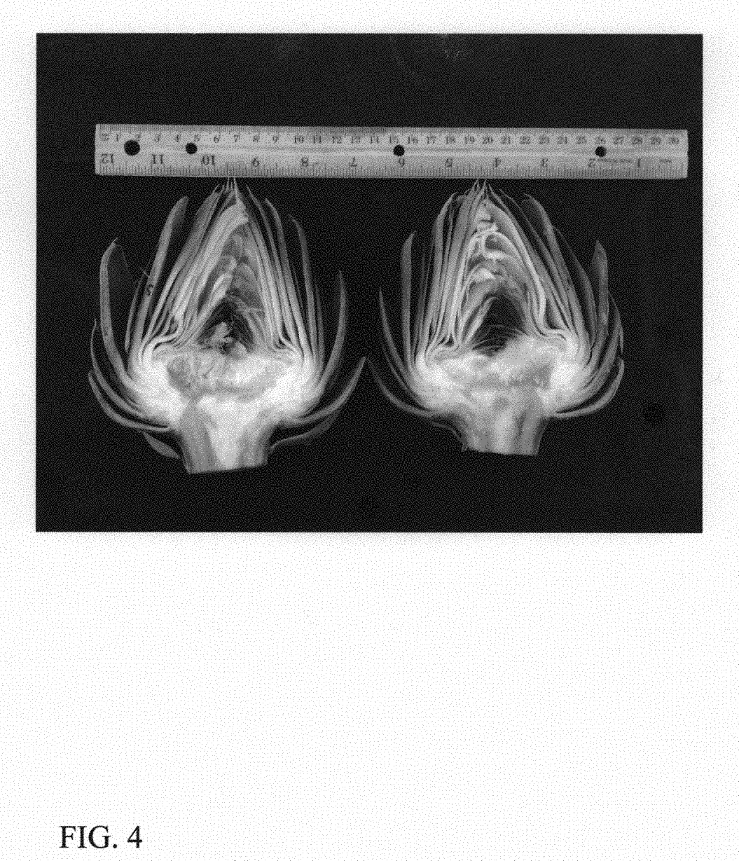

FIG. 5 shows various sized capitula (buds) of a ‘PAGA G-1’ plant.

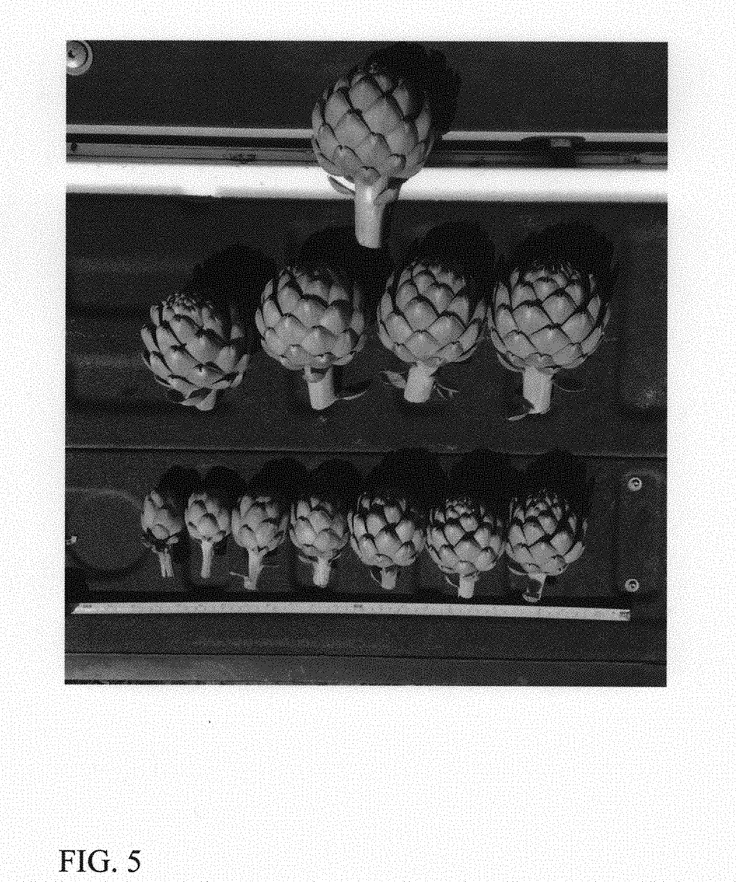

FIG. 6 shows a basal rosette leaf of ‘PAGA G-1’.

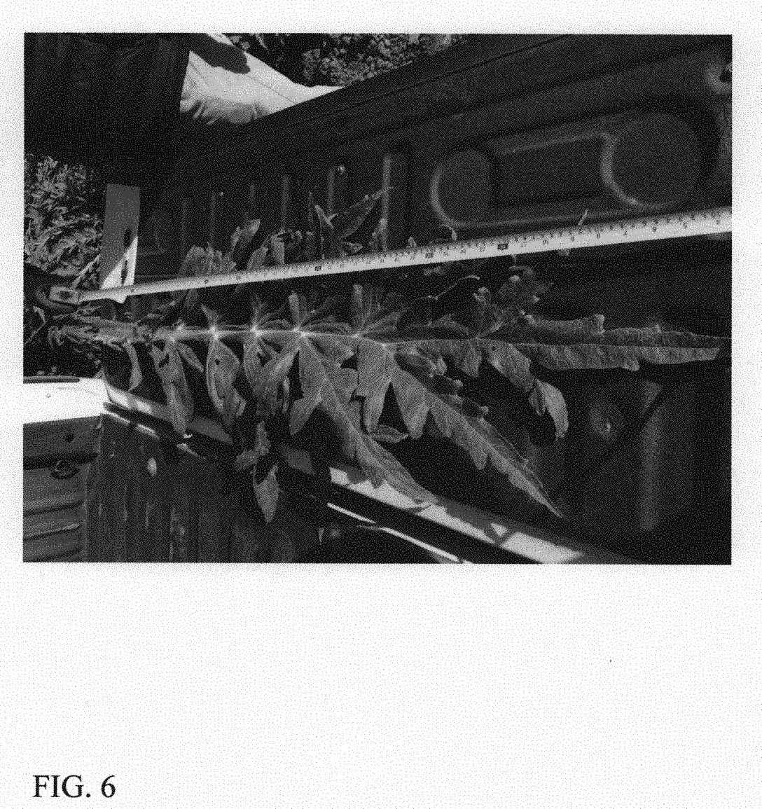

FIG. 7 shows a growing field of ‘PAGA G-1’ demonstrating its uniformity.

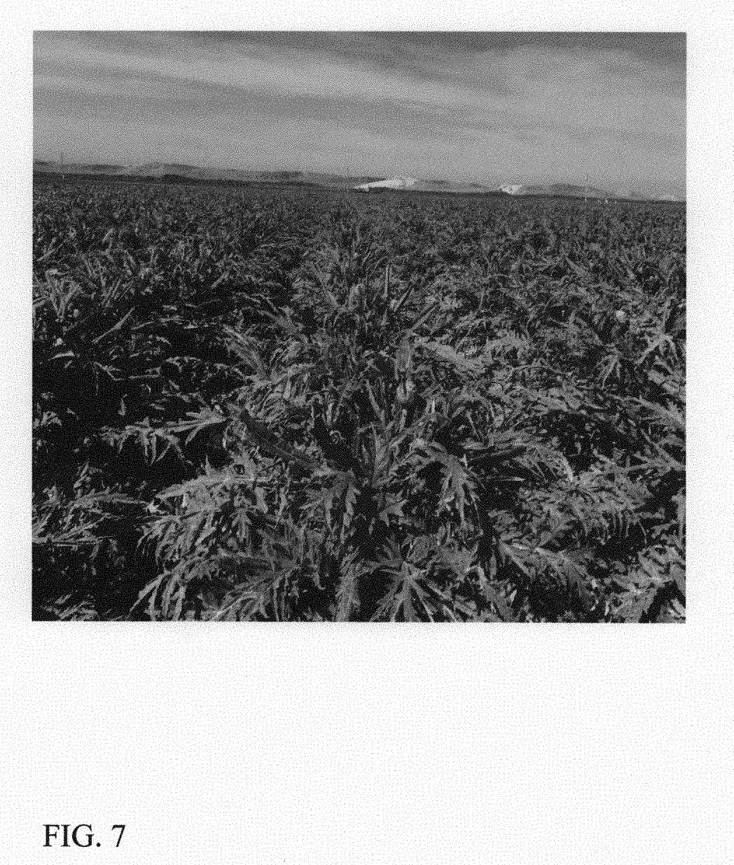

FIG. 8 shows a growing field of the parent variety ‘Green Globe’ demonstrating its lack of uniformity.

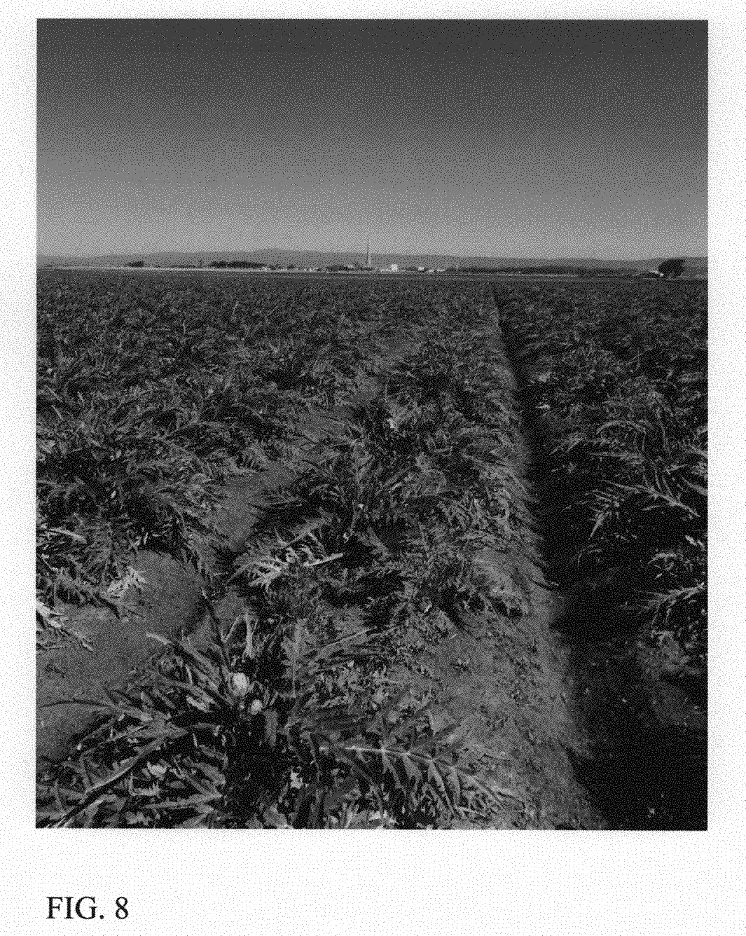

FIG. 9 shows ‘PAGA G-1’ (right) and ‘Green Globe’ (left) capitulum (bud) bases showing the differences in purple colored striations on lower bracts.

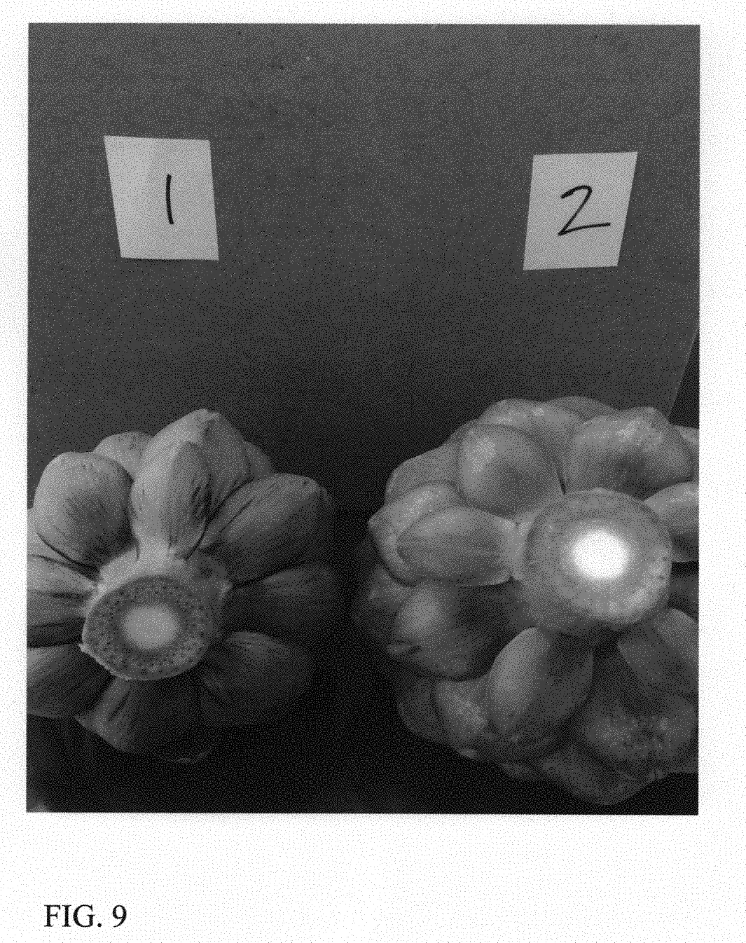

FIG. 10 shows ‘PAGA G-1’ (left) and ‘Green Globe’ (right) reproductive stems that have been stripped of leaves.

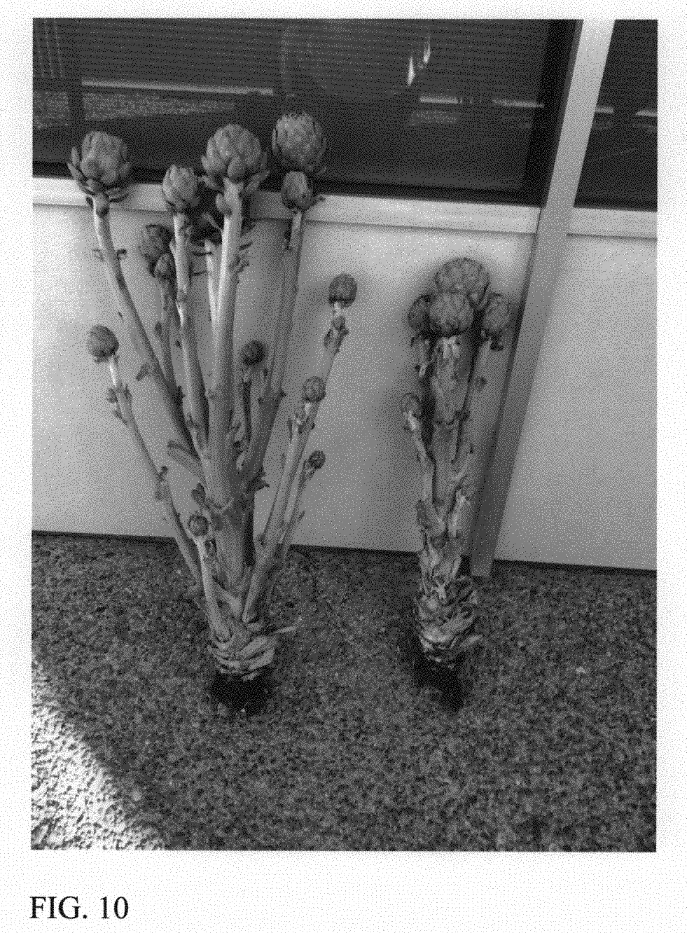

### DETAILED BOTANICAL DESCRIPTION

The following detailed descriptions set forth the distinctive characteristics of ‘PAGA G-1’. The data which define these characteristics is based on observations taken in Castroville, Calif. This description is in accordance with UPOV terminology. Color designations, color descriptions, and other phenotypical descriptions may deviate from the stated values and descriptions depending upon variation in environmental, seasonal, climatic, and cultural conditions. ‘PAGA G-1’ has not been observed under all possible environmental conditions. The botanical description of ‘PAGA G-1’ was taken from plants that were 5-6 months old. The indicated values represent averages calculated from measurements of several plants. Color references are primarily to the R.H.S. Colour Chart of The Royal Horticultural Society of London (R.H.S.) (2007 edition). Descriptive terminology follows the Plant Identification Terminology, An Illustrated Glossary, 2^{nd} edition by James G. Harris and Melinda Woolf Harris, unless where otherwise defined.
Classification:
   Taxonomic name.—Cynara cardunculus L. var. scolymus.
   Common name.—Globe Artichoke.
   Denomination.—‘PAGA G-1’.
Parentage:
   Cynara cardunculus var. scolymus L. ‘PAGA G-1’ was discovered through successive plant selections, and tissue culture propagation cycles of Cynara cardunculus L. var. scolymus variety ‘Green Globe’.
Plant characteristics table:

| General information | PAGA G-1 | Green Globe |
|---|---|---|
| Propagation Method: | Tissue culture | Crown division |
| Months of harvest: | Year round with greatest productivity | Year round with greatest |
| | February through June | productivity February through |
| | | June |
| Number of days from | Range: 165-203 Average: 190 (tissue | Range: 161-212 Average 195 |
| planting to first harvest | culture) | (crown division) |
| Duration of harvest cycle: | Average: 75 days | Average: 232 days |
| Plant | PAGA G-1 | Green Globe |
| Height range at harvest | 152.4-187.96 cm (60-74 inches) | 119.38-167.64 cm (47-66 |
| (vegetative): | | inches) |
| Height average at harvest | 167.64 cm (66 inches) | 147.32 cm (58 inches) |
| (vegetative): | | |
| Width at harvest (vegetative): | 203.2-243.84 cm (80-96 inches) | 114.3-132.08 cm (45-52 inches) |
| Growth Habit: | Upright, with horizontal spread of | Upright, with horizontal spread |
| | older leaves and shoots | of older leaves and shoots |
| Form: | Arch | Arch |
| Vigor: | Very Good | Moderate |
| Root system: | Thick and fibrous | Thick and fibrous |
| Foliage Density: | Dense, increasing as secondary | Moderately dense |
| | reproductive shoots develop | |
| Primary Reproductive | | |
| Shoot | PAGA G-1 | Green Globe |
| Height to primary (terminal) | 152.4 cm-193.04 (60-76 inches) | 106.68-137.16 cm (42-54 |
| bud top: | | inches) |
| Number of leaves | 26-35 (average 31) | 14-28 (average 20) |
| Secondary Reproductive | | |
| Shoots | PAGA G-1 | Green Globe |
| Number per plant in first | 1-5 (average 3) | 1-2 (average 1) |
| production cycle: | | |
| Height to primary (terminal) | 40.64-121.92 cm (16-48 inches) | 13.97-76.2 cm (5.5 to 30 inches) |
| bud top: | | |
| Number of leaves | 18-30 (average 23) | 3-16 (average 7.5) |
| Foliage | PAGA G-1 | Green Globe |
| Venation: | Reticulate | Reticulate |
| Leaf ratio (L/W): | Approximately 1.1-3.23 | Approximately 1.12-2.28 |
| Upper leaf surface color: | RHS 143A-143B (Green Group) | RHS 141A-141B (Green Group) |
| Lower leaf surface color: | RHS 141D (Green Group) | RHS 136D (Green Group) |
| Leaf texture: | Glabrous and slightly glossy | Glabrous and slightly glossy |
| | appearance in cool, damp conditions | appearance in cool, damp |
| | with interveinal texture. | conditions with interveinal |
| | | texture. |
| Pubescence: | Slightly tomentose on upper surface, | Slightly tomentose on upper |
| | moderately tomentose on lower. More | surface, moderately tomentose |
| | visible in dry/warm conditions. | on lower. More visible in |
| | | dry/warm conditions. |
| Petiole color: | RHS 142B-142C (Green Group) | RHS 142C-142D (Green Group) |
| Petiole and mid rib texture: | Furrowed on upper side with lower | Furrowed on upper side with |
| | side deeply ribbed with ridges | lower side deeply ribbed with |
| | running parallel to the lengthwise | ridges running parallel to the |
| | orientation. Sparsely arachnoid. | lengthwise orientation. Sparsely |
| | | arachnoid. |
| 1. Basal rosette leaves | PAGA G-1 | Green Globe |
| Shape: | Deeply pinnatifid with sparsely ciliate | Deeply pinnatifid with sparsely |
| | margins, narrowly trullate overall. | ciliate margins, narrowly trullate |
| | Narrowly acuminate apex is less acute over- | Narrowly acuminate |
| | all. than Green Globe. | apex. |
| Size: | Very large, >182.88-78.74 cm (72 × | Very large, >172.72 × 45.72 cm |
| | 31 inches) | (68 × 18 inches) |
| Distance between leaf | 6.35-11.43 cm (2 1/2-4 1/2 inches) | 6.35-11.43 cm (2 1/2-4 1/2 |
| division: | | inches) |
| Division indentation length: | 16.51-41.275 cm (6 1/2-16 1/4 | 16.51-41.275 cm (6 1/2-16 1/4 |
| | inches) | inches) |
| Petiole length: | 30.48-57.15 cm (12-22 1/2 inches) | 30.48-48.26 cm (12-19 inches) |
| Petiole width: | 3.175-9.525 cm (1 1/4-3 3/4 inches) | 3.175-6.985 cm (1 1/4-2 3/4 |
| | | inches) |
| Petiole thickness: | 0.635-1.905 cm (1/4-3/4 inches) | 0.635-1.905 cm (1/4-3/4 inches) |
| 2. Cauline leaves | PAGA G-1 | Green Globe |
| Shape: | Ensiform overall, uneven and sparse | Ensiform overall, uneven and |
| | serrations with narrowly acuminate | sparse serrations with narrowly |
| | apex. Somewhat undulate and dente | acuminate apex. Somewhat |
| | and sparsely ciliate at margins, | undulate and dente and sparsely |
| | | ciliate at margins. |
| Number of leaves: | Approximately 85 | Approximately 73 |
| Size: | 17.78-60.96 cm (7-24 inches) | 17.78-60.96 cm (7-24 inches) |
| Stem (primary reproductive | | |
| stem system) | PAGA G-1 | Green Globe |
| Length (ground level to base | 152.4 cm-193.04 (60-76 inches) | 106.68-127.0 cm (42-50 inches) |
| of terminal capitulum (bud)): | | |
| Circumference: (base of | 7.62-13.97 cm (3-5 1/2 inches) | 7.62-12.065 cm (3-4.75 inches) |
| terminal capitulum (bud): | | |
| Circumference (base of | Average: 27.94 cm (11 inches) | Average: 24.13 cm (9 1/2 |
| stem): | | inches) |
| Exterior texture: | Vertically ribbed, pubescent | Vertically ribbed, pubescent |
| Stem habit: | Upright, caulescent | Upright, caulescent |
| Pubescence: | Sparsely arachnoid | Sparsely arachnoid |
| Color: | RHS 142A-142B | RHS 142B-142C |
| Involucre bracts | PAGA G-1 | Green Globe |
| Arrangement: | Imbricate | Imbricate |
| Quantity per Capitulum | 52 outer-35 inner | 52 outer-35 inner |
| (average): | | |
| Outer bract | PAGA G-1 | Green Globe |
| Shape: | Predominately ovate with flattened | Predominately ovate with |
| | base | flattened base |
| Apex: | Retuse | Retuse |
| Margin: | Entire | Entire |
| Base: | Slightly rounded | Slightly rounded |
| Texture: | Firm, thick and fleshy | Firm, thick and fleshy |
| Color: | RHS 142A-142B (Green Group) | RHS 141C-141D (Green Group) |
| Surface (outer/inner): | Glabrous | Glabrous |
| Spinosity: | Moderate frequency, woody | Moderate frequency, woody |
| Length of spines: | 1-5 mm (0.04-0.2 inches) | 1-5 mm (0 .04-0.2 inches) |
| Striation at base of lower | Very minor-only present on the | Present on approximately 20% |
| bracts: | margins of a few lower outside bracts. | of lower outside bracts. |
| | More apparent on immature buds. | |
| Striation color: | RHS 77A (Purple-Violet Group) | RHS 77A (Purple-Violet Group) |
| Inner bract | PAGA G-1 | Green Globe |
| Shape: | Lanceolate with caudate tip | Lanceolate with caudate tip |
| Apex: | Retuse | Retuse |
| Margin: | Entire | Entire |
| Texture: | Firm, thinner than outer bract, fleshy | Firm thinner than outer bract, |
| | | fleshy |
| Base: | Slightly rounded | Slightly rounded |
| Color: | RHS 150B-150C (Green Group) | RHS 150B-150C (Green Group) |
| Surface (outer/inner): | Glabrous | Glabrous |
| Spinosity: | Finer than outer bract, more frequent | Finer than outer bract, more |
| | | frequent |
| Length of spines: | 1-2 mm (0.04-0.079 inches) | 1-2 mm (0.04-0.079 inches) |
| Capitulum (head/bud)- | | |
| when ready for harvest as | | |
| edible bud | PAGA G-1 | Green Globe |
| Color: | RHS 142A-142B (Green Group) | RHS 141C-141D (Green Group) |
| Shape: | Globose to ovoid | Globose to ovoid |
| Apex: | Broadly rounded to obtuse | Broadly rounded to obtuse |
| Base: | Truncate to rounded | Truncate to rounded |
| Form: | Tightly compact | Tightly compact |
| Texture: | Firm | Firm |
| Receptacle shape: | Concavo-convex | Concavo-convex |
| 1. Primary (Terminal) | | |
| Capitulum | PAGA G-1 | Green Globe |
| Number per primary shoot | 1 | 1 |
| Circumference: | 34.925-43.18 cm (13 3/4-17 inches) | 32.385-38.1 cm (12 3/4-15 |
| | | inches) |
| Weight: | Average 470 grams (1.03 lbs) | Average 425 grams ( .94 lbs.) |
| Height: | Average 10.795 cm (4 1/4 inches) | Average 10.16 cm (4 inches) |
| Diameter: | 12.7-15.24 cm (5-6 inches) | 11.43-12.7 cm (4 1/2-5 inches) |
| 2. Secondary capitulum | PAGA G-1 | Green Globe |
| Number per primary shoot | 7-10 | 3-5 |
| Circumference | 7.62-38.1 cm (3-15 inches) | 7.62-38.1 cm (3-15 inches) |
| Weight: | Average 321 grams (.707 lbs) | Average 237 grams ( .52 lbs.) |
| Height: | Average 9.525 cm (3 3/4 inches) | Average 8.255 cm (3 1/4 inches) |
| Diameter: | 2.54-12.7 cm (1-5 inches) | 2.54-12.7 cm (1-5 inches) |
| 3. Tertiary capitulum | PAGA G-1 | Green Globe |
| Number per primary shoot | 6-14 | 2-5 |
| Circumference | 7.62-30.48 cm (3-12 inches) | 7.62-27.31 cm (3-10 3/4 inches) |
| Weight: | Average 68 grams (0.14991 lbs) | Average 44 grams (0.0970 lbs) |
| Height: | Average 8.46 cm (3 1/3 inches) | Average 6.78 cm (2 2/3 inches) |
| Diameter: | 5.08-11.43 cm (2-4 1/2 inches) | 5.08-10.795 cm (2-4 1/4 inches) |
| Receptacle (heart)-when | | |
| ready for harvest and edible | PAGA G-1 | Green Globe |
| Shape: | Concavo-convex | Concavo-convex |
| Circumference: | 8.89-25.4 cm (3 1/2-10 inches) | 8.89-25.4 cm (3 1/2-10 inches) |
| Color: | RHS 150D (Green group) | RHS 150D (Green group) |
| Texture: | Firm | Firm |
| Size: | Very thick and wide when compared | Very thick and wide when |
| | to other globe varieties | compared to other globe |
| | | varieties |
| Peduncle | PAGA G-1 | Green Globe |
| Shape: | Columnar | Columnar |
| Length: | 2.54-101.6 cm (1-40 inches) | 2.54-76.2 cm (1-30 inches) |
| Diameter: | .635-4.445 cm (1/4-1 3/4 inches) | .635-3.81 cm (1/4-1 1/2 inches) |
| Color: | RHS 142A-142B | RHS 142B-142C |
| Strength: | Strong and rigid | Strong and rigid |
| Surface: | Glabrous, slightly pubescent with | Glabrous, slightly pubescent |
| | shallow vertical ribbing | with shallow vertical ribbing |

### COMPARISON WITH PARENTAL AND COMMERCIAL VARIETIES

When artichoke variety ‘PAGA G-1’ is compared to the parental variety ‘Green Globe’, ‘PAGA G-1’ produces larger sized secondary and tertiary buds and in greater numbers. Additionally, ‘PAGA G-1’ exhibits less phenotypic variation when grown in the field.

We claim:

1. A new and distinct variety of artichoke plant named ‘PAGA G-1’ as shown and described herein.
